(19) United States
(12) Patent Application Publication
(10) Pub. No.: US 2025/0127571 A1
(43) Pub. Date: Apr. 24, 2025

(54) METHOD FOR DETERMINING A DESTRUCTIVE TEARING OF AN EYE TISSUE, COMPUTER PROGRAM, OPHTHALMOLOGICAL ANALYSIS APPARATUS AND OPHTHALMIC SURGICAL SYSTEM

(71) Applicant: Carl Zeiss Meditec AG, Jena (DE)

(72) Inventors: Matthias Hillenbrand, Oberkochen (DE); Rebekka Peter, Oberkochen (DE); Ludwig Haide, Oberkochen (DE); Steffen Peikert, Oberkochen (DE); Daniel Reichard, Oberkochen (DE); Johannes Fauser, Oberkochen (DE)

(73) Assignee: Carl Zeiss Meditec AG, Jena (DE)

(21) Appl. No.: 18/918,939

(22) Filed: Oct. 17, 2024

(30) Foreign Application Priority Data
Oct. 23, 2023 (DE) 102023128998.5

(51) Int. Cl. A61B 34/10

(52) U.S. Cl. CPC A61B 34/10; A61B 2034/104; A61B 2034/105; A61B 2034/107

(57) ABSTRACT

A method for determining a destructive tear in real tissue of a real eye by simulation includes providing an evaluation unit with at least one item of information characterizing the real tissue of the eye. Using the evaluation unit, the method includes using the information to create an at least two-dimensional digital tissue model of the eye tissue. The method includes simulating a tearing action on the tissue using a simulated surgical instrument and/or a simulated point where the instrument acts on the tissue, particularly for actions that could potentially cause tearing during a real ophthalmic surgical procedure. The evaluation unit is then provided with information regarding the reaction of the tissue model to the simulated action. Using the evaluation unit, the method includes using the reaction information and the simulated tearing action to determine a potential tear line in the real tissue.

### TECHNICAL FIELD

One aspect of the disclosure relates to a method for determining a destructive tearing of a tissue in an eye by simulation. A further aspect of the disclosure relates to a computer program. Moreover, one aspect of the disclosure relates to an ophthalmological analysis apparatus. Furthermore, one aspect of the disclosure relates to an ophthalmic surgical system.

### BACKGROUND

In the case of ophthalmic surgical procedures on the eye, for example when removing a crystalline lens from the eye and replacing this crystalline lens with an implanted intraocular lens, this crystalline lens is removed from the capsular bag of the eye. To this end, it is necessary to open the capsular bag. In this context, producing an opening on an anterior capsular bag wall for the purposes of removing the crystalline lens is known.

Opening the capsular bag at its anterior capsular bag wall, for example by the action of laser radiation, and thereby creating a hole in this anterior capsular bag wall is known.

In contrast to such procedures, mechanical tearing tools such as needles or forceps, for example, are known. These tools are introduced into the anterior chamber of the eye through an incision in the cornea, created in advance by means of a surgical knife, and the anterior capsular bag wall is lacerated by these mechanical tearing tools by way of the direct action on the anterior capsular bag wall. This creates an appropriate opening in the anterior capsular bag wall in order to then be able to remove the crystalline lens from the capsular bag. By way of example, the crystalline lens can be removed by phacoemulsification.

In comparison with non-mechanical tearing tools, as are exemplified by lasers, for example, these mechanical tearing tools are widespread, particularly for reasons of costs.

However, inaccuracies when forming the tear are also possible in the case of these mechanical tearing tools. This may arise by virtue of an inaccurate rhexis, especially with regard to the tear geometry and tear position relative to the capsular bag, arising on the anterior capsular bag wall on account of the handling of the mechanical tearing tool by the surgeon. This may result in inaccurate and unwanted directions of the rhexis and/or undesirably large or undesirably narrow forms of the openings on the anterior capsular bag wall. If such disadvantages occur and if rhexis portions that reach as far as a circumferential edge of the anterior capsular bag wall arise, this may result in a disadvantage to the effect of the intraocular lens to be subsequently implanted no longer being able to be held securely and reliably, especially not in symmetric and centered fashion, in the capsular bag. It may even be the case in individual cases (typically ˜1%) that, especially on account of mechanical action, the anterior capsular bag lacerates so far that it is no longer possible at all to fasten an intraocular lens therein and thereby additional surgical complexity and a higher risk of further complications arise. In particular, these alternatives generally yield a less optimal refractive result for the patient. Even though these disadvantages arise in the case of mechanical tearing tools, these tools are very widespread in medical procedures on account of low costs and a relatively fast production of an opening in the anterior capsular bag wall. In particular, a mechanical tearing tool can be better embedded in the surgical processes than a laser, meaning a faster overall procedure.

Current surgical procedures are usually performed manually by medical staff. However, telemanipulated robots, too, are already known in surgery. Additionally, the degree of automation is increased in surgical procedures, and there is increased assistance by such telemanipulated robots.

It generally holds true that properties of the tissue to be treated differ from patient to patient. Thus, surgical staff being very knowledgeable in the art currently is an essential advantage with regard to selecting the correct method and performing the surgical procedure such that it is appropriate for the properties of the tissue to be operated on. However, this also applies to telemanipulated robot-assisted methods, in which surgical staff control the robot through the various steps of the method. In this context, automated surgery is very dependent on in-depth understanding of the properties and behavior of the tissue, especially in the eye. This with regard to being able to distinguish between intended and unintended alterations to the tissue during the automatically performed operation and the planning thereof.

### SUMMARY

The problem addressed by the present disclosure is that of developing a method, a computer program, an ophthalmological analysis apparatus and an ophthalmic surgical system, in which the understanding with regard to individual real tissue is increased and the repeatability of a surgical procedure for different tissues is improved and more adapted to individual circumstances as a result.

This problem is solved by a method, a computer program, an ophthalmological analysis apparatus and an ophthalmic surgical system.

One aspect of the disclosure relates to a method, in particular a computer-implemented method, for determining a destructive tear in a real tissue of a real body part, in particular a real eye, by simulation, preferably including the following steps:
a) in particular providing an evaluation unit, in particular, with at least one item of information characterizing the real tissue of the eye;
b) in particular using the evaluation unit to create, in particular, an at least two-dimensional digital tissue model for the tissue on the basis of the information from step a);
c) in particular simulating a tearing action on the tissue on the basis of a surgical instrument created by simulation and/or a point created by simulation where the surgical instrument acts on the tissue, especially in the case of at least a potentially specifically tearing action on the tissue during a real ophthalmic surgical procedure;
d) in particular providing the evaluation unit with information regarding a reaction of the tissue model on account of the simulated action in step c);
e) using the evaluation unit to determine an at least potential tear line in the real tissue on the basis of the reaction in accordance with step d) and the simulated tearing action in accordance with step c).

Such a method now makes it possible to also provide an electronic analysis system having at least one evaluation unit with, in particular, real information regarding an eye to be treated. A particular advantage of the method is that the provided information relating to the eye to be treated for example allows this analysis system to also determine, by simulation, desired and necessary destructive tearing of a tissue, for example of an anterior wall of a capsular bag. In particular, destructive tearing means that the tissue is not torn from an edge, which is easier to control. In particular, it means that tearing starts at a starting location distant from an edge of the tissue. In the case of a capsular bag wall, which is an example of a tissue, the edge can be the region in which the capsular bag is connected to the zonular fibers. Therefore, tearing preferably starts from a hole or an opening created in the tissue. The hole can be an elongate line for example created by cutting, i.e. cutting through, the tissue. In particular, tearing is started at one end of this line-like separation line in the tissue, in particular at a radially outer end. In the context of cutting, the tissue separation is implemented by a cutting tool such that the tool always makes contact where the tissue has not yet been separated. Thus, the further cutting separation is always implemented directly where the tool acts directly on the tissue. This is not the case for tearing. This is because the tissue is gripped with a tool or instrument at a gripping location in the case of tearing, and the tissue is subsequently tom open. Since the gripping location in this case does not change during a tearing process, the gripping location moves increasingly further away from the front tearing point where the transition between tear line and not yet torn tissue is situated. Precisely this makes accurate tearing of the tearing line more difficult. The method proposed above is particularly advantageous precisely as a result thereof. Such a procedure is envisaged, precisely when tearing open the capsular bag, in particular an anterior wall of the capsular bag.

What is a particularly advantageous in the case of the method is that it simulates the movement of the modeled tissue which occurs on account of a specific action on the tissue during an operative procedure that cannot be avoided. A great advantage of the proposed method is that the resultant movement of the tissue, i.e. in particular the movement as a whole and/or the deformation of the tissue at least in partial regions, is simulated in three dimensions. In particular, this can also be implemented if the modeled tissue is modeled only in two dimensions. As a result, an adequate amount of outlay, in particular also computational outlay, is required to obtain a precise statement with regard to the behavior of the tissue. In particular, dynamic processes can also be simulated quickly and precisely thereby. Thus, the model allows prediction of this movement of the tissue by simulation in particular. Hence, this model can also be based on a movement prediction model for predicting a movement of the tissue.

In particular, the model is created as a digital tissue model individual to the eye. In particular, this means that there is not the same global tissue model for various eyes but that this tissue model is only created in a manner adapted as precisely as possible to the eye to be observed and analyzed, i.e. the entire eye or a specific part thereof, which is relevant to the procedure. Additionally, the distinction of the locality of a specific tissue, for example due to variations in the tissue thickness or earlier damage, can for example be taken into account during the analysis if this is advantageous for the procedure.

Such a digital tissue model individual to the eye can therefore differ from eye to eye, in particular in view of the number and/or the type of parameters describing the tissue model.

The tissue can be a single tissue or a tissue system formed from a plurality of tissues, in particular also different tissues, of the eye. In particular, it is a capsular bag of an eye.

The method allows a three-dimensional consideration precisely when tearing open a capsular bag, and so the tear line is more accurately determinable and predictable in the very dynamic procedure and behavior of the capsular bag. This has significant advantages for the actual subsequent process on the eye during the operative procedure.

It is also possible that the determined tear line is provided as output information by the evaluation unit. It is also possible in this case that the evaluation unit creates a control signal on the basis of the tear line determined by simulation, said control signal rendering at least one surgical instrument and/or a display unit controllable, especially during an operative intervention on this real eye. In particular, it is also possible that the at least one determined potential tear line is provided, in particular on a surgical system, as auxiliary information during an operative procedure on the eye.

In an exemplary embodiment, a simulated prediction of a reaction of the modeled tissue which occurs on account of the simulated action in step c) can be created in step d), in particular using the evaluation unit. This prediction can then be provided. It is also possible that the provision of the reaction is implemented on the basis of a machine-trained algorithm.

It is possible that a reaction is a movement, preferably a three-dimensional movement, of the modeled tissue. Provision can also be made for a reaction to be a stress of the modeled tissue.

In an exemplary embodiment, a stress threshold value for the tissue is specified by the tissue model and/or estimated by the evaluation unit, at least with regard to one location of the modeled tissue. A stress threshold value can be specified and/or estimated for the stress in the tissue and/or for the tear resistance and/or the pressure resistance. It is possible that this stress threshold value is determined by the evaluation unit on the basis of the tissue model, or this value is specified by the tissue model itself. Such a parameter allows for a more accurate prediction of the effect on the tissue and its tearing behavior, and critical situations can be identified better and, as a consequence, be analyzed and predicted better. At least one such threshold value for at least one portion of the modeled tissue in particular allows the mechanical strength thereof to be characterized more accurately, in particular also in order to be able to better simulate and predict the start of tearing and/or the tear direction. In particular, supercritical states of the tissue, which are unwanted irreversible and destructive states, can also be avoided in the context of the force effects.

It is possible that the stress threshold value is determined using tests and/or preoperative data. Optionally, at least one measurement can be implemented at discrete points, and the determination on the basis of measurement information can then be implemented by interpolation with the evaluation unit.

In an exemplary embodiment, a start of the tissue tearing is determined by the evaluation unit on the basis of the stress threshold value and a force effect, as created by simulation in step c), on the tissue model. In particular, a start or further tearing of the tear line is determined, in particular on the basis of the locally and/or temporally determined start of tearing. In particular, a three-dimensional movement of the surgical instrument can also be determined more accurately on the basis thereof.

In one exemplary embodiment, the potential tear line is created in three dimensions. This is a further advantageous exemplary embodiment. This is because this allows the tear line to be predicted particularly exactly and realistically. Very complex mechanical influences that have an influence on the shape and/or direction of the tear line arise precisely on account of the diverse movements of the tissue, for example a wall of a capsular bag, in three dimensions when a force acts on the said tissue. Thus the three-dimensional simulation of this tear line has particular advantages with regard to being able to increase all facets of the exactness and recognizability of the line profile.

In an exemplary embodiment, a three-dimensional movement of the surgical instrument is specified by simulation in step c) as simulated tearing effect. As a result, the effect of specific movements of an instrument on the specific tissue, in particular the tearing behavior, can be examined by simulation. Precisely the interplay between a movement of the instrument on the tissue and the individual tissue characterized by the tissue model allows precise simulation of the action/reaction principle in order to identify when a tear occurs, where it occurs and how it progresses.

As tissue, a tissue wall is created as a tissue model, in particular as an at least two-dimensional surface, in one exemplary embodiment. A two-dimensional surface can be created easily and with little outlay. Furthermore, it is sufficient for specific tissues, for example a capsular bag, in particular a wall of the capsular bag. Even by simulation, it provides a very realistic structure for the real tissue. However, it is also possible that the tissue is simulated as a three-dimensional object. For example, modeling can be implemented using a thin element (surface) which also has a three-dimensional contour. It is also possible to model the tissue as a volume model.

The tissue model is preferably created as a mesh with mesh nodes and polygonal surfaces as mesh elements. A simple structure of the tissue can be simulated in this way. Then again, a very flexible tissue is able to be modeled by the multiplicity of polygonal surfaces, and so even a small portion of the tissue can be simulated very accurately in view of its three-dimensional movement, both on its own and/or in relation to other portions. Hence, the three-dimensional simulated movement of the modeled tissue as a whole and also in portions thereof can be implemented in various ways, and hence be simulated more realistically. It is particularly advantageous in this case that tears in the tissue can also be simulated accurately, in particular precisely along side lengths of the polygonal surfaces.

For example, the polygonal surfaces can be triangles or quadrilaterals. By preference, a side length of a polygonal surface can be between 0.005 and 0.15, in particular between 0.08 and 0.12 mm. This is advantageous, in particular, in the case of a triangle, in particular an equilateral triangle.

By preference, an edge of the modeled tissue is simulated as stationary within the simulation when a force effect on the tissue is simulated. This contributes to a higher exactness and a more accurate prediction of the tear line, especially in the case of a wall, for example like an anterior wall of a capsular bag.

In particular, the simulated tearing action on the tissue is performed by a front end or tip of a surgical instrument created by simulation. As a result of this simulated direct action of the tip, the mesh is moved by simulation in three dimensions and/or at least deformed in portions by simulation.

By preference, a force effect of the instrument on the modeled tissue is simulated in step c) and the stresses in the modeled tissue arising in the process are determined in three dimensions in particular using the evaluation unit. A propagation direction of the tear line is determined on the basis of the stresses by the evaluation unit.

The tissue, in particular a tissue wall of a tissue, is preferably characterized as linear elastic material during the modeling. This enables simple modeling which nevertheless is very accurate, especially when determining a tear line. However, nonlinear models may also form the basis of tissue modeling. Then accuracy is prioritized over calculation speed.

In an exemplary embodiment, in the tearing of the tissue, as determined by simulation, a start of the tearing is followed by the use of the evaluation unit to determine a temporal change of a gripping location, at which the instrument is in engagement with the tissue, to a different gripping location of the tissue and/or the use of the evaluation unit to determine a spatial change of a gripping location, at which the instrument is in engagement with the tissue, to a different gripping location of the tissue, this depending on the stresses in the modeled tissue and/or depending on the modeled force effects of the instrument and/or depending on at least a partial element of the modeled tissue. Precisely when tearing open an anterior wall of a capsular bag, this also allows a particularly accurate prediction, by simulation, of the coupling between the instrument and the lacerated capsular bag during the further tearing. Since the location at which the instrument on the lacerated flap of the wall moves away from the anterior site of the tear which continually migrates as the laceration advances, this gripping location and the moving away in particular has an influence on the course of the tear line. This can be analyzed and predicted by simulation using this exemplary embodiment. This also allows a determination of situationally dependent and individual temporal and/or local changes in the gripping location, which are required to design the tear line to the best possible extent.

In step a) of an exemplary embodiment, real information regarding the tissue of the eye measured during a surgical procedure is provided as characterizing information and/or real information regarding the tissue of the eye measured during a preliminary examination, especially immediately preceding the surgical procedure, is provided as the characterizing information. For example, this can be determined using test functions and/or experiments on non-critical sites of the real tissue.

Thus, for example, information determined preoperatively, which preferably also characterizes a real reaction of the real tissue in the eye, and/or, preferably, information determined intraoperatively, which preferably also characterizes a real reaction of the real tissue of the eye, are provided for step a) in one exemplary embodiment.

Moreover, the characteristic information can also relate to the type and/or strength of a tissue defect, for example, in particular of a defect intended to be at least improved by way of the surgical procedure. In particular, it is also possible to take account of (bio)mechanical properties. Moreover, this may also be based on geometry information for at least partial elements of the real tissue. In this context, partial elements can be for example the cornea, the capsular bag, zonular fibers, the crystalline lens, the pupil, etc. or portions thereof. For example, geometric parameters can be a thickness, a diameter, a length, a distance or the like.

In addition to that or in an alternative, living being-specific information for the living being, to whom the eye belongs, and/or phenotypical information and/or geometric information regarding the eye can also be provided to the evaluation unit as characterizing information, and/or biomechanical parameters and/or the mass and/or the elasticity of the eye, in particular of the real tissue of the eye can be provided as characterizing information. This allows diverse specifications regarding the degree of the creation of the tissue model that is individual to the eye and/or the individual depth of detail of at least portions of the tissue to be modeled.

In particular, it is possible that, as already presented above, the information provided in step a) is ascertained by test functions. These test functions are preferably performed on the individual tissue of the patient. In particular, mechanical and biomechanical properties should be included among the tissue properties. For example, this includes the elasticity and the strength, such as the tensile strength, the flexural strength and the torsional strength. However, tissue properties can also be thermal and thermomechanical properties. Biological and optical properties are also examples of tissue properties. In this case, some of the properties can also be geometry dependent and/or direction dependent.

Moreover, further additional patient data such as age, sex, race, metabolism, metabolic factors such as diabetes or blood pressure, for example, or known ailments, medicaments, such as blood thinners or cortisone, for example, and/or disorders can be used as provided information. These can then also be taken into account by the analysis system in the creation of the digital tissue reaction model and/or for the prediction of a potential reaction of the real tissue. Standard surgical instruments or specific test instruments can be used to perform the test functions. Additionally, sensor systems such as a surgical microscope or an intraoperative OCT (optical coherence tomography) system can be used to read the reactions of the tissue and/or tool to the applied test functions. A tool-integrated sensor can also be used.

A creation of a tissue model individual to the eye means in particular that this is not based on a global standard model for tissue which is defined globally by specific parameters and in which only parameter values allow for a certain amount of individualization of the tissue. Instead, in the context of the application, a creation individual to the eye in particular means that the number of parameters which describe the tissue and which should form the basis for the creation of the tissue model are selected depending on the situation and on an individual basis, in particular by the analysis system itself. In addition to that or in an alternative, provision can then also be made for the number and/or the type of partial elements of a tissue or tissue system, which should form the basis for the digital tissue model to be created, to be selected on an individual basis, in particular by the analysis system itself. In this way, there can be significantly greater flexibility in view of the creation of a digital tissue model, at least in respect of the underlying parameters and/or the underlying partial elements of a tissue. In another exemplary embodiment, it is also possible that the creation of a tissue model individual to the eye can be based, in particular by the analysis system itself, on functional links between the selected parameters and/or links between the selected partial elements. Hence, the individual partial elements of the tissue model to be created and/or parameters to be considered can also be linked on an individual basis.

In one exemplary embodiment, this analysis system can also use artificial intelligence, for example, to in this way create a tissue model individual to the eye. Thus, in this context, this analysis system may also comprise a neural network. This also allows the analysis system to train itself continuously and improve accordingly. In particular, the complexity of such a creation of a digital tissue model individual to the eye can also be improved on the basis of a neural network. This is because individual action/reaction processes on the basis of corresponding selected parameters and/or selected partial elements for the tissue model to be created can then be adapted and matched in even more improved fashion to the situation of the real tissue.

This improves or increases the individualization and needs-based creation of a digital tissue model which is as realistic as possible.

In an exemplary embodiment, at least the creation of the tissue model in accordance with step b) and/or step c) is performed using an FEM (finite element method) approach. This method approach is advantageous, in particular in relation to the aforementioned advantages, for precisely these steps.

It is also an essential aspect that the analysis system also creates prediction information, including at least the potential tear line determined by simulation and/or a determined trajectory of a surgical instrument in particular, by way of the simulated modeling. Thus, a prediction result in particular for a tear line in the tissue, in particular in an anterior wall of the capsular bag, is performed on the basis of the model using this evaluation unit, in particular the analysis system. This prediction information or the prediction result then preferably provides an example of auxiliary information provided by the analysis system. As a result, this renders possible a very exact and individual model-based analysis of a specific tissue which should be treated during a surgical procedure. This process and the prediction result as auxiliary information allow both medical staff and an automated surgery system to perform this operative procedure more accurately and more safely (with lower complication rates).

Providing the result of the prediction as auxiliary information can mean displaying this auxiliary information on a display apparatus, in particular an ophthalmic surgical system, in one exemplary embodiment. However, it is also possible that this provision is implemented on a control unit of an ophthalmic surgical system, in order to control at least one surgical instrument by the control unit on the basis of this auxiliary information. In particular, this is advantageous if the ophthalmic surgical system is a telemanipulated robot-assisted system or a semiautonomous or fully autonomous robotic system. The control unit can be an evaluation unit or comprise the latter.

Within the scope of providing the information in step a), it is possible that these real reactions are in this context caused or provoked by test functions. In particular, this can be carried out on a real eye prior to the operative procedure. In another exemplary embodiment, it is also possible that, in addition to that or in an alternative, such test functions are performed during an operative procedure and, in that case, real reactions of the real tissue are also provoked during the operative procedure. These test functions can be performed either on the tissue that is removed or on tissue that remains untouched.

Hence it is possible that the auxiliary information is created prior to the surgical intervention, and in particular also completed in that case, in one exemplary embodiment. In another exemplary embodiment, it is possible that, in addition to that or in an alternative, the creation of auxiliary information is performed during an operative procedure. It is also possible that such test functions are performed on dummy eyes or test eyes. These can also be provided as real, physical models. It is also possible that such a test function is created by simulation on such a digital eye created by simulation, and that a digital reaction occurs in that case. Thus, this particularly advantageously allows a prediction to be made on the basis of this auxiliary information, with regard to how the real tissue will react. Hence, the surgical procedure can be adapted thereto in a much more individual fashion. In particular, this makes it possible to better avoid unwanted tissue reactions or better avoid medical staff or a surgical robot being surprised by a tissue reaction during the surgical procedure. Since sensitization for medical staff and/or the ophthalmic surgical system is achieved by way of the auxiliary information, there can be an improved reaction to possible expected tissue reactions, also in anticipatory fashion, and the surgical procedure can be adapted better thereto. This also improves the operation result.

For example, an eye tissue can be the cornea, the capsular bag, zonular fibers, the crystalline lens, the pupil, etc. In particular, it is an anterior wall of the capsular bag. A tissue can also be a membrane on the retina.

Tearing can be desired tearing required for the operative procedure. However, it can also be unwanted tearing that should be avoided.

In one exemplary embodiment, additional auxiliary information that differs from the auxiliary information is created with the aid of the evaluation unit. This can extend the information base created and provided by the analysis system.

In one exemplary embodiment, a type of use of a surgical instrument to be performed for the ophthalmic surgical procedure, in particular a movement guidance, is created as additional auxiliary information. In addition to that or in an alternative, a surgical instrument to be used for the ophthalmic surgical procedure can be selected or proposed as additional auxiliary information. In addition to that or in an alternative, a type of implant provided for implantation in the eye can be created as additional auxiliary information. For example, such an implant can be an intraocular lens. Hence, such first additional auxiliary information is information that does not directly relate to information about the tissue or to information or actions for the operative procedure, dependent thereon or to be derived therefrom; instead, it in particular represents information regarding the objects that are advantageous for the operative procedure.

In one exemplary embodiment, a location on the eye for placement and/or introduction of a surgical instrument and/or a plan for performing an ophthalmic surgical intervention and/or a warning message is created as further additional auxiliary information, in particular second additional auxiliary information. This is also additional advantageous information which is advantageous for medical staff or else advantageous within the scope of a surgical procedure performed in at least partly automated fashion. This is because this also enables the performance of more adequate operative procedures in the case of different eyes and hence different tissues and tear lines resulting therefrom, and so complications or disadvantageous operation results can be significantly reduced. For example, a surgical robotic system could perform a procedure on the basis of the prediction, analyze the tear line created by simulation, and adapt the further process on the basis of the additional auxiliary information generated therefrom.

In one exemplary embodiment, additional auxiliary information is created on the basis of the auxiliary information using the evaluation unit. This performs a cascaded automated creation of helpful information for the preparation of an operative procedure and/or for the performance of an operative procedure. Consequently, the at least one evaluation unit can intelligently create an information tree in needs-based fashion. This allows the number of items and/or type of auxiliary information to be designed more flexibly and more variably. Hence, the creation of the type and/or the number of items of helpful information can also be matched here to the eye to be treated.

The auxiliary information can be output on an output unit in one exemplary embodiment. The output unit can be a constituent part of the ophthalmic surgical system. Hence, the auxiliary information and/or the additional auxiliary information can also be displayed for medical staff and can be perceived by them. An output unit can be a display. However, it is also possible that the output unit is a constituent part of a surgical microscope. Hence, the auxiliary information and/or the additional auxiliary information can also be overlaid into the field of view of medical staff working with the surgical microscope. In addition to that or in an alternative, the output can also be implemented as an acoustic signal, for example as a speech signal, or as a haptic signal.

A further aspect of the disclosure relates to a computer program comprising commands which, when the program is executed by a computer, cause the latter to carry out the method according to the aforementioned aspect or an advantageous exemplary embodiment. In this context, the computer can be a computing unit or evaluation unit. It can be a constituent part of an ophthalmic surgical system. The computing unit can be designed separately from a control unit. However, it can also be a constituent part of the control unit.

A further aspect of the disclosure relates to a medical analysis apparatus, in particular an ophthalmological analysis apparatus. It comprises at least one evaluation unit. It may also comprise at least one memory. This analysis apparatus or the analysis system is designed to carry out a method according to the aforementioned aspect or an advantageous exemplary embodiment thereof. In particular, the method is carried out with the ophthalmological analysis apparatus. This ophthalmological analysis apparatus can be a constituent part of an ophthalmic surgical system.

A further aspect of the disclosure relates to an ophthalmic surgical system having at least one surgical instrument. The system is designed to perform, in particular plan and/or adapt, a surgical procedure, by preference at least on the basis of the tear line determined and provided by the method according to the aforementioned aspect or an advantageous exemplary embodiment thereof. In addition to that or in an alternative, the system can be designed to offer and/or select a first operating method or a second operating method, which differs therefrom, preferably at least on the basis of a tear line determined by the method according to an aforementioned aspect or an advantageous exemplary embodiment thereof. In this context, it is also possible that a recommendation is output, in particular for medical staff. It is also possible to offer or select a decision in this respect, both in binary and parametric fashion. The ophthalmic surgical system can be designed for phacoemulsification. In addition to that or in an alternative, it can be designed, in particular, to perform capsulorhexis on an eye. Retinal peeling is also possible.

A further independent aspect relates to a method for operating an ophthalmic surgical system, in particular including the following steps:
in particular providing at least one item of auxiliary information, including at least a tear line and/or a trajectory for a system unit such as a surgical instrument of the system in particular, created by the method according to the aforementioned aspect or an advantageous exemplary embodiment thereof;
in particular operating at least one system unit, for example at least one surgical instrument and/or an output unit and/or operation observation equipment, of the system on the basis of the auxiliary information provided, in particular during a surgical procedure on an eye.

A piece of operation observation equipment can be a surgical microscope or an OCT system. The OCT (optical coherence tomography) system can also comprise sensors directed at an operating region from a greater distance.

In particular, the operation of the system unit or functional unit can be a selection and/or setting of operating parameters and/or a movement, for instance a robot-assisted movement or a guided movement, of the system unit.

In particular, the system unit can be controlled in this case, preferably during the surgical procedure. This also enables a higher degree of automation and also a high degree of safety during an operative procedure.

In particular, in addition to that or in an alternative, operating methods can be proposed and/or selected on the basis of the auxiliary information, in particular the determined tear line, in particular by the system, and/or a surgical procedure can be planned. To this end, it is for example also possible to determine the type and/or number of system units required or possibly required.

In particular, the repeatability of surgical procedures can be increased by the proposed method, the computer program, the ophthalmological analysis apparatus and the ophthalmic surgical system, by virtue of these being based on an improved understanding of the patient-specific tissue properties. What this also achieves is that medical staff, such as surgeons, are assisted in the selection of the most suitable surgical method for the individual patient and their individual tissue properties. Moreover, the medical staff can be assisted in the performance of the surgical procedure by a better understanding of the tissue properties. In particular, the medical staff can be assisted by assistance functions, such as warning systems or trajectory recommendations for guiding a surgical tool as well. However, the method is also particularly advantageous in the case of at least semiautonomous, in particular also fully autonomous, robot-assisted surgical methods. This can then be based on the determined tear line as input parameter, for example for the trajectory planning algorithms or other algorithms that influence the behavior of the robotic system. In this context, the ophthalmic surgical system, as specified above, can also be an at least semiautonomous surgical robotic system.

As already explained above, the aforementioned auxiliary information can be output. In one exemplary embodiment, the further interpretation can be left to the specialist medical staff. In another exemplary embodiment, the auxiliary information can be processed on part of the system, in particular by way of algorithms and/or reference data. As a result, there can be an improved preparation which is then made available to the medical staff to allow for a simpler interpretation by the latter. For example, in this context, the auxiliary information and/or a measure for the reliability of the auxiliary information can also be based on a traffic light system.

Moreover, the auxiliary information might also be processed by using algorithms and/or reference data, in order to provide assistance functions for the medical staff in particular. In this context, assistance functions can be force warnings or surgical instructions. Moreover, it is possible that the auxiliary information is processed by algorithms and/or reference data for providing inputs for planning a trajectory with regard to the trajectory guidance of a surgical instrument. This can also be implemented for at least semiautonomous partial robotic tasks and corresponding trajectory planning algorithms. Moreover, in the case of a telemanipulated system, it is possible to define certain regions into which the surgical element cannot be guided despite corresponding specifications by the surgeon. Additionally, the auxiliary information can be processed further by algorithms and/or reference data in order to provide recommendations regarding the best suitable access for a surgical instrument on the real eye and/or for selecting and/or recommending the most suitable tools or surgical instruments and/or implants.

Especially the combination of the provided information, which characterizes a real reaction of the real tissue of the eye, with preoperative data provides advantageous base information for the system-side creation of the tissue model individual to the eye. This also enables a very individual design of the model and, on the basis of this model, a generation of the prediction, very individual to the eye, of a tear line in particular and/or, resulting therefrom, the creation of auxiliary information. A particularly exact validation and adaptation of the operative procedure is rendered possible thereby.

In particular, the concept can be provided for a body part in general, and not only for an eye. A body part of a living being is, in particular, a body part that is elastically deformable under an action thereon. This can be a body-internal body part, and hence be located completely within the body of a living being. However, this can also be a body part visible from the outside. The body part can be arranged on or in the head.

In that case, the analysis apparatus can also be a medical analysis apparatus in general. In that case, the system can be a surgical system in general.

Further features of the disclosure are evident from the claims, the figures and the description of the figures. The features and combinations of features mentioned in the description above and the features and combinations of features mentioned in the description of the figures below and/or shown only in the figures can be used not only in the respectively specified combination but also in other combinations, without departing from the scope of the invention. The invention shall thus also be considered to include and disclose configurations of the disclosure that are not shown and elucidated explicitly in the figures but arise from and can be created through separate combinations of features from the configurations elucidated. Disclosure shall also be considered to extend to configurations and combinations of features that thus do not have all the features of an independent claim as originally worded. Disclosure shall additionally be considered to extend to embodiments and combinations of features, in particular by virtue of the embodiments explained above, which go beyond or depart from the combinations of features set out in the dependency references of the claims.

The concrete values indicated in the documents for parameters and indications concerning ratios of parameters or parameter values for the definition of exemplary embodiments of the apparatus should be considered to be concomitantly encompassed by the scope of the disclosure even in the context of deviations, for example on account of measurement errors, system faults, DIN tolerances, etc., which means that explanations relating to substantially corresponding values and indications should also be understood thereby.

### BRIEF DESCRIPTION OF THE DRAWINGS

Exemplary embodiments of the disclosure will be explained in detail below with reference to schematic drawings, in which:

FIG. 1 shows a schematic illustration of an exemplary embodiment of an ophthalmic surgical system having an exemplary embodiment of an ophthalmological analysis apparatus;

FIG. 2 shows an illustration of a lacerated tissue, in particular an anterior wall of a capsular bag;

FIG. 3 shows an illustration of an exemplary embodiment of a modeled tissue created by simulation from a mesh;

FIG. 4 shows a perspective illustration of the tissue in accordance with FIG. 3, certain areas of which have been destructively lacerated by simulation;

FIG. 5 shows a partial illustration of a modeled tissue with a modeled tear line and information regarding stress on the tissue determined by simulation;

FIG. 6 shows a partial illustration of tissue with a tear line, modeled as a mesh; and

FIG. 7 shows the illustration in accordance with FIG. 6 with a redesign of the mesh in the region of the tear.

### DETAILED DESCRIPTION

In the figures, identical or functionally identical elements are given the same reference signs.

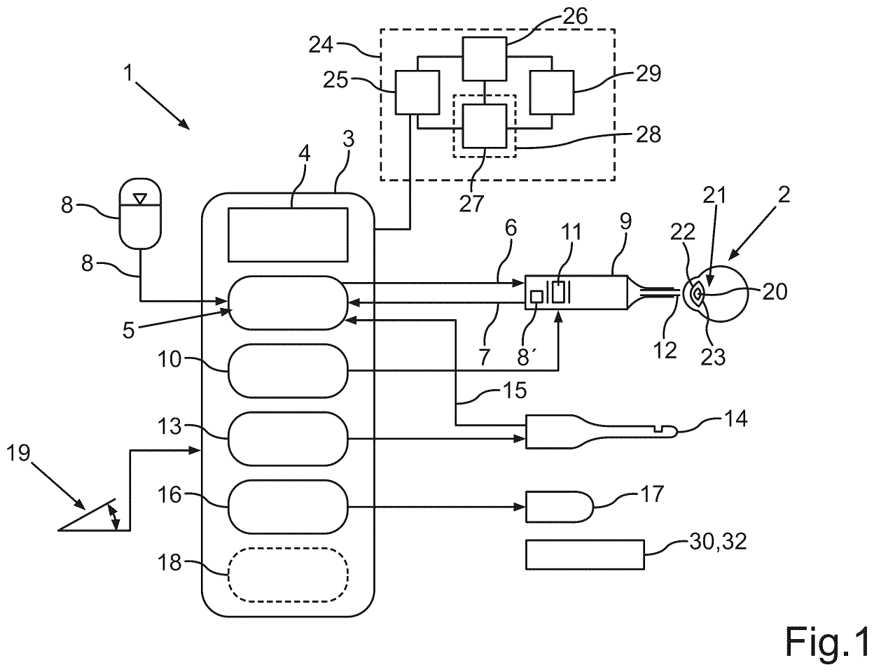

FIG. 1 shows a schematic illustration of an ophthalmic microsurgical system or an ophthalmic surgical system 1, at least for phaco surgery on an eye 2. The illustration in accordance with FIG. 1 shows some components of the system 1 symbolically for the simplified explanation of the basic general functionality of the system 1. The system 1 can also be used for membrane peeling on a retina.

The system 1 preferably comprises an equipment unit 3, which for example can be a trolley or the like. By preference, an operating unit 4 is arranged in or on the equipment unit 3. For example, this operating unit 4 might comprise a user interface, an input unit such as a keyboard or the like and a display unit such as a monitor or a display. Furthermore, a fluidic system 5 comprising a pump and a control unit for controlling the pump and connected components is preferably arranged in the equipment unit 3. The fluidic system 5 comprises an irrigation apparatus 6 having an irrigation branch and an aspiration apparatus 7 having an aspiration branch. The irrigation apparatus 6 comprises a container 8 for rinsing solution, for example a BSS solution, which is an irrigation fluid which is guided to a phaco handpiece. The phaco handpiece is an ophthalmic surgical handpiece 9. In particular, it is a constituent part of the ophthalmic surgical system 1. The aspiration apparatus 7 is likewise connected to the ophthalmic surgical handpiece 9. The handpiece 9 is an example of a surgical instrument. By preference, the system 1 comprises further surgical instruments. For this purpose, provision can also be made of a tool for lacerating the capsular bag of an eye. A surgical instrument for polishing the capsular bag might also be provided and be a constituent part of the system 1. These examples should be understood not to be exhaustive.

In an alternative embodiment, provision can be made for the ophthalmic surgical system 1 to comprise a tank 8′ (FIG. 1) that is separate from the container 8. In such an embodiment, the cooling fluid provided to cool the incision is provided separately from the irrigation apparatus 6 and separately from the container 8. The tank 8′ can be arranged separately from the handpiece 9 and, for example, can be connected to the handpiece 9 via a line, such as a tube connection.

In a further embodiment, provision can be made for the separate tank 8′ to be arranged in the handpiece 9. It can be arranged in the handpiece 9 in non-destructively non-detachable or non-destructively detachable fashion. Provision might also be made for a first separate tank 8′ to be arranged in the handpiece 9 and for a further separate tank 8′ to be arranged outside of the handpiece 9. This further tank 8′ outside of the handpiece 9 can be fluid-conductively connected to the tank 8′ arranged in the handpiece 9.

Moreover, the equipment unit 3 comprises an ultrasonic unit 10 in particular, the latter being designed to excite an oscillation of a piezo-component 11 in the ophthalmic surgical handpiece 9, by means of which a hollow needle 12 of the ophthalmic surgical handpiece 9 is excited to oscillate. Further, the equipment unit 3 comprises a control unit 13, in particular. The control unit 13 can also be designed to control a vitrectomy handpiece 14, which, in particular, might be a constituent part of the ophthalmic surgical system 1. The vitrectomy handpiece 14 is an example of a surgical instrument. By preference, the vitrectomy handpiece 14 is also connected to the fluidic system 5, in particular by an aspiration line 15. Moreover, a further control unit 16 can be provided, the latter controlling a preferably present further surgical instrument 17, for example for diathermy. Moreover, the system 1 and, in particular, the equipment unit 3 can comprise further modules and control units and systems, which are represented symbolically by the unit 18. This also comprises further internal units, and also peripheral equipment. Moreover, the ophthalmic surgical system 1 preferably comprises a foot control panel 19, which is connected to the equipment unit 3, in particular to communications devices and control units of the equipment unit 3.

A cutting tool for cutting open a capsular bag of the eye 2 can be a further surgical instrument 30.

A tearing tool for lacerating a capsular bag of the eye 2 can be a further surgical instrument 32. This especially holds true if the said capsular bag, in particular an anterior wall of the capsular bag, is locally cut open up by the cutting tool 30 such that the wall is subsequently torn open further at that point.

The eye 2 can be a real, living eye. It comprises a crystalline lens 20 arranged in a capsular bag 21. The capsular bag 21 comprises an anterior capsular bag wall 22 and a posterior capsular bag wall 23. The eye 2 can be a real eye of a human or an animal. However, it can also be a dummy eye. In this embodiment, it might have been taken from a dead organism and be a dummy eye made of biological material. However, the dummy eye might also be manufactured artificially, for example, and be formed at least in part from plastics, for example. However, it might also be an eye that is only displayed on an electronic visual display in a simulation. In this embodiment, the eye 2 can be represented 2-dimensionally or 3-dimensionally. The dummy eyes or the eye created by simulation also include(s) the usual components, in particular a lens 20 and a capsular bag 21.

FIG. 1 also shows an exemplary embodiment of an ophthalmic surgical apparatus, which is an ophthalmological analysis apparatus 24 in particular. The ophthalmological analysis apparatus 24 can be a constituent part of the ophthalmic surgical system 1. However, the ophthalmological analysis apparatus 24 can also be a separate apparatus. It can be used both during the actual real surgical procedure on a real eye 2 and, for example, for the purpose of training or educating medical staff. The ophthalmological analysis apparatus 24 can also be used as a separate unit without the system 1, especially for training or educational purposes. It can be an analysis system.

The ophthalmological analysis apparatus 24 preferably comprises at least one input unit 25. The latter can be a keyboard. However, it can also be a touch-sensitive input field. However, in addition to that or in an alternative, the input unit 25 can also be designed for voice input.

The ophthalmological analysis apparatus 24 preferably comprises at least one evaluation unit 26. In particular, the evaluation unit 26 is also designed to evaluate the input information which was input by way of the input unit 25. The ophthalmological analysis apparatus 24 preferably comprises an optical display unit 27. This optical display unit 27 can be a separate electronic visual display. However, the optical display unit 27 can also be a constituent part of a surgical microscope 28, for example. The display unit 27 is an example of an output unit.

The ophthalmological analysis apparatus 24 preferably comprises at least one capturing device 29. For example, the capturing device 29 can be an image creating unit such as a camera. In particular, this image creating unit is sensitive in the spectral range that is visible to humans.

In addition to or instead of the aforementioned use purposes, the ophthalmological analysis apparatus 24 can also be designed purely as an ophthalmological analysis system. In such a configuration, operation results of an already completed real operation can also be evaluated and analyzed retrospectively. For example, a surgical microscope (stereo cameras) with or without an OCT (optical coherence tomography) system can be provided for 3-D imaging. For example, in addition to or instead of this, a Brillouin microscope might also be provided, which can render stresses in the tissue visible.

With the ophthalmological analysis apparatus 24, it is possible to carry out a method for creating auxiliary information for an ophthalmic surgical procedure on a real eye.

Moreover, the ophthalmological analysis apparatus 24 can be used to carry out a method, in particular a computer-implemented method, for determining a destructive tear in a real tissue in a real eye 2 by simulation. The information determined in the process, in particular a tear line determined by simulation, can be an example of auxiliary information. In particular, the auxiliary information can include this determined tear line.

FIG. 2 shows a schematic illustration of a portion of the anterior capsular bag wall 22. Here, this anterior capsular bag wall 22 is already partly shown in the lacerated state. The anterior capsular bag wall 22 has a bounding edge 22a. As evident from FIG. 2, the laceration did not start at this edge 22a. Rather, the laceration of the capsular bag 21, in particular of this anterior capsular bag wall 22, started at a distance and spaced apart from this edge 22a. To this end, in particular, a surgical instrument 30 was used to puncture this not yet destroyed capsular bag 21, in particular the non-destroyed anterior capsular bag wall 22, in a manner so as to cut through the latter at a distance from this edge 22a. This puncture by the instrument 30 cuts through by way of a cutting action, and so an elongate separation or an elongate hole 31 is formed in the anterior capsular bag wall 22. This separation line created by a cutting action is straight or substantially straight, as is also evident from FIG. 2. This separation line has a radially inner end 31a and a radially outer end 31b. Using this attained state as a starting point, there can subsequently be a laceration, in contrast to the capsular bag wall 22 being cut open as implemented previously. To this end, for example and by preference, the symbolically depicted instrument 32 is used such that this instrument 32 engages with the tissue at a contact point, in particular a gripping location 33, especially at the radially outer end 31b. Movement of this instrument 32 causes the tissue, the anterior capsular bag wall 22 of the capsular bag 21 in this case, to be lacerated such that a tear line 34 arises, a tear line portion of which is shown here. As shown in this intermediate state shown in FIG. 2, in which the anterior wall 22 has not yet been lacerated completely, the gripping location 33 remains unchanged during a tear procedure, and so, during a continual tear procedure, this gripping location 33 becomes increasingly distant from a respective anterior and migrating end 35 of the continually increasing tear line 34. A lacerated tissue flap 40 of the capsular bag wall 22 is shown.

In particular, this tissue to be lacerated in this example generally is a very thin wall. It is inherently dimensionally unstable. This means that, when considered alone, it cannot be set up or maintained in dimensionally stable fashion. In such tissues in particular, the described procedure during the laceration is unstable, and so the course of the tear line 34 is very difficult to predict. In particular, the course of the tear line 34 can also very quickly assume an unexpected course on account of very diverse influencing parameters. This harbors disadvantages since the subsequent implantation of the intraocular lens in the remaining capsular bag 21 can be made more difficult by an imprecise or undesired laceration, and hence an undesirable course of the tear line 34. It is therefore of utmost importance to be able to create the tear line 34 as exactly as possible.

It is therefore particularly advantageous if such a determination, in particular a prediction of at least a potential tear line 34 in the real tissue, can be made on the basis of the computer-implemented method described hereinbelow. In this method, the following steps are preferably carried out:
a) providing an evaluation unit 26 with at least one item of information characterizing the real tissue of the eye 2;
b) using the evaluation unit 26 to create an at least two-dimensional digital tissue model 36 for the tissue on the basis of the information from step a);
c) simulating a tearing action on the tissue using, or on the basis of, a surgical instrument 30, 32 created by simulation and/or a point created by simulation where the surgical instrument (30, 32) acts on the tissue, especially in the case of at least a potentially specifically tearing action on the tissue during a real ophthalmic surgical procedure;
d) providing, in particular making a simulated prediction of, a reaction that takes place on account of the simulated action in step c), in particular a three-dimensional movement and/or stress, of the tissue model 36 for the evaluation unit 26 and/or using the evaluation unit 26.

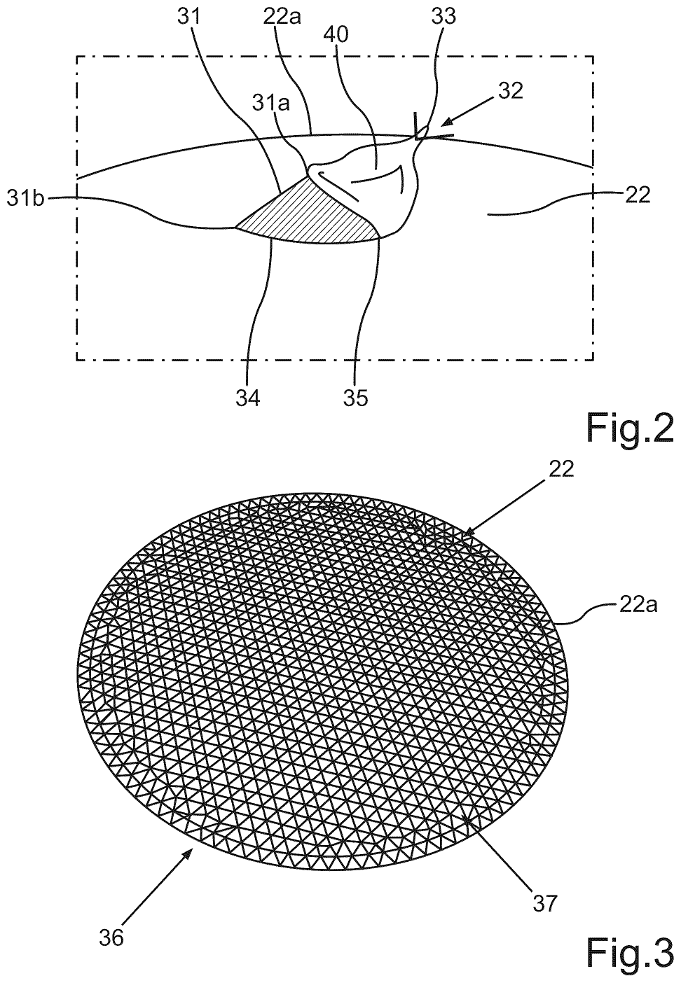

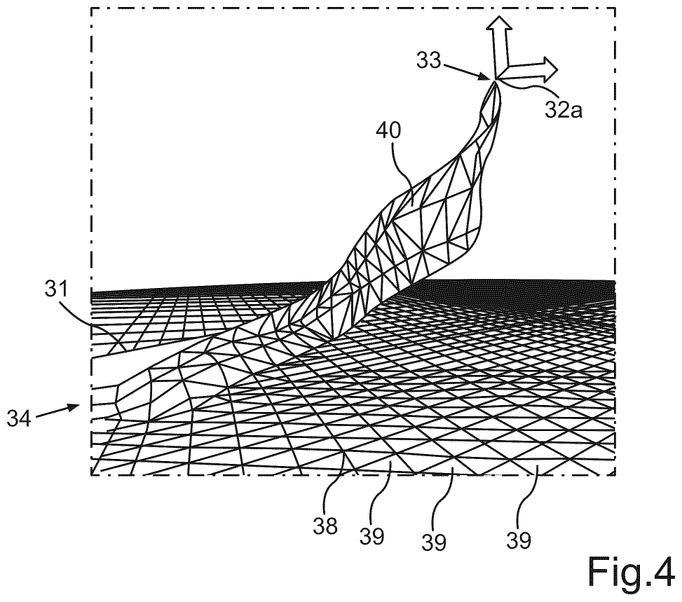

In the presented exemplary embodiment, provision is made for an FEM approach to be taken, especially for at least steps b) and/or step c). In this context. FIG. 3 shows an exemplary embodiment in which the tissue, in particular the anterior wall 22 of the capsular bag 21 in this case, is created as a digital tissue model 36. As evident here, this anterior wall 22 is simulated as a mesh 37 by a simulation using the FEM modeling approach. In this case, the mesh 37 has a multiplicity of mesh nodes 38 (FIG. 4) and polygonal surfaces, especially triangular surfaces in this case, as mesh elements 39. For the sake of clarity, only some of these mesh elements 39 that are directly adjacent to one another are provided with the corresponding reference sign. In an exemplary embodiment, provision can be made for this tissue to be created as a two-dimensional surface. However, provision is made in particular for this surface to be able to deform three-dimensionally and/or be moved as a whole, and for this to be able to be simulated in the model. Hence, deformations and/or movements of this surface, which is two-dimensional in this case, are represented in three dimensions. A thin 3-D volume of the tissue is also possible.

Starting with the explanations already given in relation to FIG. 2, the laceration of the anterior capsular bag wall 22 by way of this simulation method is depicted in FIG. 4 using a simplified illustration. Both the separation line 31 and the onset of laceration, as explained and presented accordingly in FIG. 2, with a tissue flap 40 already created in that case are shown. By preference, the digital tissue model 36 shown in FIG. 3 is created with a diameter of between 8 mm and 12 mm, in particular between 9 mm and 11 mm. When the tear line 34 is created by simulation, provision is preferably made for the edge 22a to remain stationary. In particular, this simulative method then simulates the movement of a tip 32a of the instrument 32. This tip 32a is the part of the instrument 32 in direct engagement with the tissue. A point of action is also formed by this location. In an alternative to that or in addition, this point of action can also be created by simulation.

By preference, this digital tissue model specifies a stress threshold value for this tissue at at least one location of the modeled tissue, the capsular bag wall 22 in this case. In addition to that or in an alternative, it is also possible that such a stress threshold value is estimated by the evaluation unit 26 and/or ascertained on the basis of preoperative/intraoperative tests.

By preference, a start of this tissue tearing is determined by the evaluation unit 26 on the basis of this stress threshold value and a force effect, as created by simulation in step c), on the tissue model 36. In particular, this is implemented on the basis of the locally and/or temporally determined start of the tearing; in particular, a three-dimensional movement of the instrument 32, in particular of the tip 32a, and/or a migration of the point of action is also simulated in the process.

In particular, the stress threshold value and/or the movement of the tissue can also be determined in location-dependent fashion. Since the thickness of the tissue, in particular of the capsular bag wall, may be different at different locations, these location-dependent stresses and/or movements also allow the implementation of more accurate and hence more realistic simulations.

It is particularly advantageous if the potential tear line 34 determined by simulation is created in three dimensions.

In an exemplary embodiment, a mass for this tissue is also specified within the simulative method. In this case, a mass of between 4 mg and 6 mg, in particular 5 mg, is preferably specified in the case of an anterior capsular bag wall 22 of a capsular bag 21. This membrane or the tissue is preferably modeled as a linear elastic material. It might also be modeled in nonlinear or viscoelastic fashion.

In particular, a force effect of the instrument 32 on the modeled tissue is simulated in step c) and the stresses in the modeled tissue, specifically the tissue model 36 shown here, arising in the process are determined in three dimensions in particular using the evaluation unit 26. A propagation direction of the tear line 34 is determined on the basis of the stresses by the evaluation unit 26. In the tearing of the modeled tissue, as determined by simulation, a start of the tearing is followed by use of the evaluation unit 26 to determine a temporal change of a gripping location 33, at which the instrument 32 is in engagement with the capsular bag wall 22, to a different gripping location of the capsular bag wall 22, this depending on the stresses in the modeled tissue, i.e. the tissue model 36, and/or depending on the modeled force effects of the instrument 32 and/or depending on at least a partial element of the tissue model 36. The evaluation unit 26 can also determine a spatial change of a gripping location 33, at which the instrument 32 is in engagement with the capsular bag wall 22, to a different gripping location of the capsular bag wall 22. The instrument 32 is connected to the tissue model 36 in this simulation. If the instrument 32 created by simulation is moved, the mesh 37 is deformed in three dimensions and a stress is indicated. In particular, an iterative simulation with discrete time steps is carried out. By preference, the algorithm is performed at each considered time interval of the tear creation. Following this process, the mesh nodes 38 are updated, especially on the basis of the force effects. This is implemented using numerical integration in particular. In particular, the positions relative to one another are updated in the process. In particular, the same procedure then restarts in order to carry out a corresponding tear algorithm for the subsequently provided time window or time interval. The stresses are preferably calculated using the FEM approach. The stress tensors of at least a plurality of mesh elements 39, in particular of all mesh elements, are calculated on the basis of the positions of the mesh nodes 38. These stress tensors are used to preferably determine von Mises stresses in these mesh elements 39, in particular in all of these mesh elements 39.

In a simplification, provision can be made for the further course of a tear line at these aforementioned individual time intervals to always only be determined using the tear obtained to this point and/or the tear line created to this point as a starting point. Therefore, this simplification allows the consideration of only those mesh elements 39 located in the adjacent surroundings of this already present tear end point or tear end location of the already existing and created tear line for the respective further determination of the next tear line portion. In particular, it is then preferably possible to only consider these mesh elements 39 for the further analysis.

The tear is continued or a further tear line portion of the tear line 34 is created by simulation whenever these von Mises stresses exceed a specified stress threshold value, especially in at least one of the mesh elements 39 around this already obtained endpoint 35 of the tear line portion of the tear line 34 created and determined in advance.

The course or the direction of this tear line portion depends in particular on the direction of the fundamental stresses in the mesh elements 39 around this already obtained endpoint 35 or tear end location of the already determined tear line portion 34. By preference, the direction vector is scaled in length, in particular to a length of less than 0.2 mm, in particular between 0.10 mm and 0.18 mm, and in particular 0.15 mm. This value is preferably used as it is preferably longer than a longitudinal side of a mesh element 39 but then again still is small enough to ensure the exactness of the further course of the tear line 34.

By preference, a tolerance can be provided between the scaling value and the side length of a mesh element 39. This is advantageous on account of the direction of development or direction of advance, since the said direction is projected on the mesh 37 and therefore increased in length.

After this direction of development is calculated, in particular by the evaluation unit 26, the direction vector is projected onto the mesh 37 in an advantageous exemplary embodiment. Then, the mesh 37 is split along the path as given by the course. For example, this can be effected using Delauney triangulation.

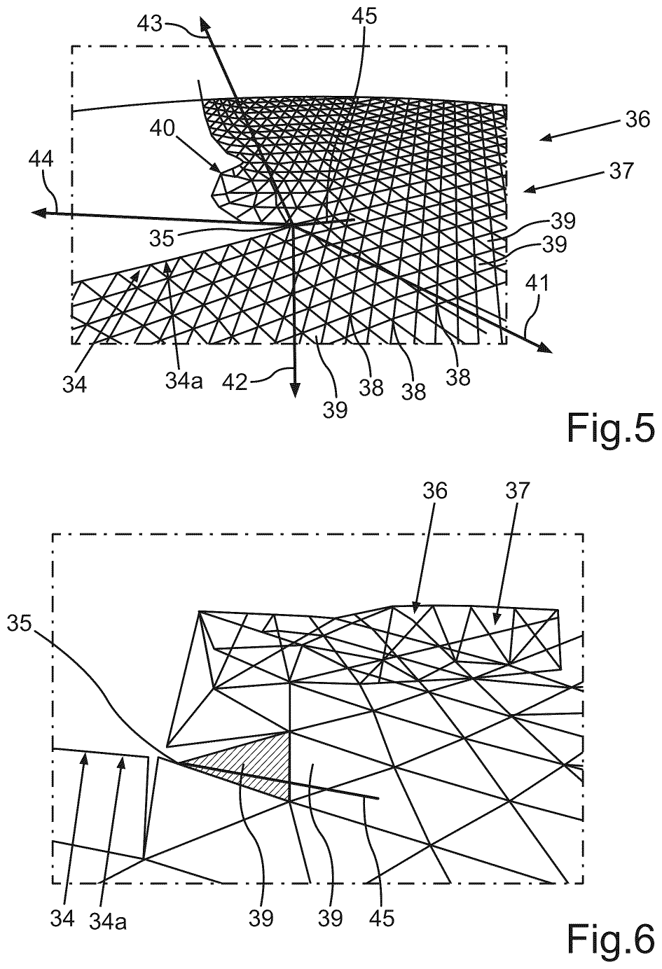

In this context, FIG. 5 shows such a procedure by way of example. A first tear line portion 34a of the tear line 34 to be determined has already been created in this portion of the mesh 37. At a current front endpoint 35 in the laceration direction, the stresses in the mesh elements 39 are analyzed around this front endpoint 35, as already mentioned. In FIG. 5, the basic or fundamental stress directions in these mesh elements 39 are depicted by way of example by the stress arrows 41, 42, 43 and 44. As a result therefrom, FIG. 5 also shows that a prediction or determination of the further course of the tear line 34 is made on the basis of these stress directions 41 to 44, which in terms of number and orientation should be understood as merely exemplary and which are intended to only symbolize the fundamental procedure, and is shown by the portion 45 depicted by simulation. This portion 45 therefore represents an example of a further tear line portion potentially following the already created tear line portion 34a. In particular, an endpoint 35 present or a tear end location reached in each case is thus extracted in each case. Such an extraction can be implemented both in 2-D and in 3-D. It is also possible that all images created by simulation can be extracted, this being advantageous for the visualization of the process created by the simulation. In particular, such an extraction can be implemented at each time step, in particular each time interval, of the simulation.

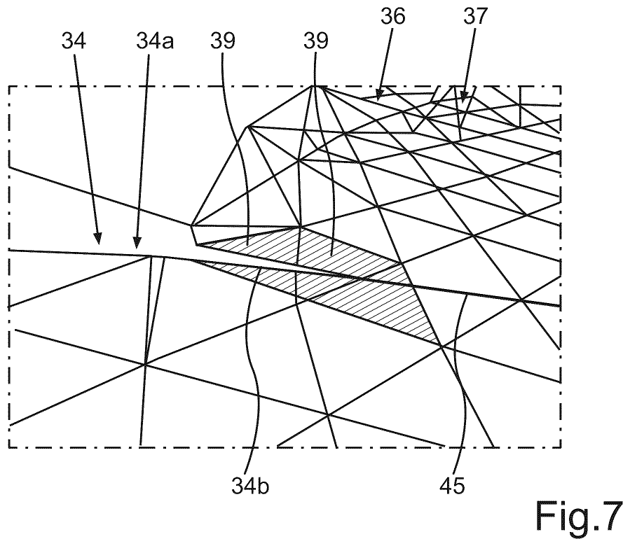

With regard to the preferred procedure already explained above, it is also possible to elaborate on FIGS. 6 and 7 hereinafter. FIG. 6 once again shows the situation in accordance with FIG. 5, with this being used as a starting point, wherein the mesh elements 39 which are affected by the further course of the tear line 34 in accordance with the prediction line or the further potential portion 45 and which should be tom open are shown in this case. In that case, they can be also color coded. Using this is a starting point, FIG. 7 then shows that these affected mesh elements 39 have in part already been ripped open and that the method, in particular the FEM approach, brings about a rearrangement of the mesh elements 39. In particular, a reorientation and alignment of the mesh elements 39 is implemented to this end, in such a way that a redistribution and/or reorientation and/or reformation of the mesh elements 39 is implemented in this region, as shown in FIG. 7. Therefore, FIG. 7 for example also shows a further created tear line portion 34b as part of the portion 45, which follows the tear line portion 34a.

In particular, the ophthalmic surgical system 1 can be provided with the information, in particular including at least the tear line 34 determined by simulation, which was determined from the aforementioned computer-implemented method, in particular by simulation. In particular, control signals for a control unit of the ophthalmic surgical system 1 can be created on the basis of this information provided. In particular, at least one system unit or functional unit, for example a surgical instrument, of the ophthalmic surgical system 1 can be controlled on the basis of these control signals.

In particular, a method for operating an ophthalmic surgical system 1 is rendered possible on the basis thereof in general.

Moreover, however, it is all possible to carry out a method for planning a surgical procedure using a surgical system 1, in particular an ophthalmic surgical system, on the eye 2. Within the scope of this planning, it is for example possible to plan a trajectory along which a surgical instrument is guided on or in the eye 2 during the surgical procedure, on the basis of at least one item of auxiliary information, in particular including at least the above-determined tear line, regarding the real tissue of the real eye 2. In particular, this can be implemented using the evaluation unit 26. In addition to that or in an alternative, a surgical procedure can be adapted on the basis of at least this aforementioned determined auxiliary information, in particular including at least the determined tear line 34. In addition to that or in an alternative, this planning on the basis of at least one item of auxiliary information as determined above, in particular including at least the tear line 34 determined by simulation, can be used to select a first operating method or an at least second operating method, which differs therefrom, in particular to offer and/or select this by way of the system 1 itself.

In addition to that or in an alternative, it is also possible that appropriate information is output on an output unit, for example the display unit 27, during this planning on the basis of this auxiliary information determined by simulation, in particular including at least the tear line 34 determined by simulation.

1. A computer-implemented method for determining a destructive tear in a real tissue of a real eye by simulation, the method comprising:
providing an evaluation unit with at least one item of information characterizing the real tissue of the eye;
using the evaluation unit to create an at least two-dimensional digital tissue model for the tissue on the basis of the information characterizing the real tissue of the eye;
simulating a tearing action on the tissue on the basis of a surgical instrument created by simulation and/or a point created by simulation where the surgical instrument acts on the tissue;
providing the evaluation unit with information regarding a reaction of the tissue model on account of the simulated tearing action;
using the evaluation unit to determine an at least potential tear line in the real tissue on the basis of the reaction of the tissue model and the simulated tearing action.

2. The method of claim 1, wherein a stress threshold value for the tissue is specified by the tissue model and/or estimated by the evaluation unit, at least with regard to one location, in particular a current endpoint of a tear line portion of the modeled tissue.

3. The method of claim 2, wherein the evaluation unit is used to determine a start of a tearing of the tissue on the basis of the stress threshold value and a force effect on the tissue model created by simulating the tearing action, wherein the tear line is determined on the basis of the locally and/or temporally determined start of tearing.

4. The method of claim 1, wherein the potential tear line is created in three dimensions.

5. The method of claim 1, wherein, as simulated tearing effect, a three-dimensional movement of the instrument is specified by simulation and/or a point where the surgical instrument acts on the tissue is specified by simulation.

6. The method of claim 1, wherein, as tissue, a tissue wall is created as tissue model as a surface, wherein the tissue model is created as a mesh with mesh nodes and polygonal surfaces as mesh elements.

7. The method of claim 1, wherein a force effect of the instrument on the modeled tissue is simulated and the stresses arising in the modeled tissue in the process are determined three-dimensionally using the evaluation unit, wherein a future direction of propagation of the tear line is determined on the basis of the stresses by the evaluation unit.

8. The method of claim 1, wherein, in the tearing of the tissue, as determined by simulation, a start of the tearing is followed by the use of the surgical instrument with the evaluation unit to determine a temporal change of a gripping location, at which the instrument is in engagement with the tissue, to a different gripping location of the tissue and/or a spatial change of a gripping location, at which the instrument is in engagement with the tissue, to a different gripping location of the tissue, this depending on the stresses in the modeled tissue and/or depending on the modeled force effects of the instrument and/or depending on at least a partial element of the modeled tissue.

9. The method of claim 1, wherein real information regarding the tissue of the eye measured during a surgical procedure is provided as characterizing information and/or real information regarding the tissue of the eye measured during a preliminary examination is provided as characterizing information and/or living being-specific information for the living being, to whom the eye belongs, and/or phenotypical information and/or geometric information regarding the eye is provided as characterizing information, and/or biomechanical parameters and/or the mass and/or the elasticity of the eye, in particular of the real tissue of the real eye is provided as characterizing information.

10. The method of claim 1, wherein at least the creation of the tissue model is performed using an FEM approach.

11. A method for planning a surgical procedure on an eye using a surgical system,
wherein a trajectory along which a surgical instrument is guided during the surgical procedure on or in the eye is planned using an evaluation unit, on the basis of at least one tear line in the real tissue of the real eye, as determined by simulation in accordance with the method of claim 1,
and/or
wherein a surgical procedure is performed on the basis of at least one tear line in the real tissue of the real eye, as determined by simulation in accordance with the method of claim 1,
and/or
wherein a first operating method or a second operating method, which differs therefrom, is selected on the basis of at least one tear line in the real tissue of the real eye, as determined by simulation in accordance with the method of claim 1,
and/or
wherein the at least one tear line in the real tissue of the real eye, as determined by simulation in accordance with the method of claim 1, is output on an output unit.

12. A computer program comprising commands that, upon execution of the program by a computer, cause the latter to carry out the method of claim 1.

13. An ophthalmological analysis apparatus having at least one evaluation unit, wherein the analysis apparatus is designed to carry out the method of claim 1.

14. An ophthalmic surgical system having at least one surgical instrument,
wherein the system is designed to carry out a surgical procedure on the basis of at least one tear line in the tissue, as determined by the method of claim 1,
and/or
wherein the system is designed to offer and/or select a first operating method or a second operating method, which differs therefrom, on the basis of at least one tear line in the tissue, as determined by the method of claim 1,
and/or
wherein the system is designed to control at least one system unit of the system on the basis of at least one tear line in the tissue, as determined by the method of claim 1,
and/or
wherein the system is designed to display on operation observation equipment of the system in three dimensions, as an overlay on a current real image of the eye, the tear line in the tissue, as determined by the method of claim 1.
